(19) United States
(12) Patent Application Publication
(10) Pub. No.: US 2025/0205023 A1
(43) Pub. Date: Jun. 26, 2025

(54) VISUALIZING AND MODELING A DENTAL PROSTHESIS

(71) Applicant: KOMBO MEDICAL SOLUTIONS UG, Goslar (DE)

(72) Inventors: Kim KUBIACK, Goslar (DE); Carsten NITSCH, Goslar (DE)

(21) Appl. No.: 18/869,306

(22) PCT Filed: May 4, 2023
(86) PCT No.: PCT/EP2023/061808

§ 371 Date: Nov. 26, 2024

(30) Foreign Application Priority Data
May 30, 2022 (DE) 10 2022 113 599.3
Feb. 24, 2023 (DE) 10 2023 104 576.8

(51) Int. Cl. A61C 13/00; G16H 30/40; G16H 50/50

(52) U.S. Cl. CPC A61C 13/0004; G16H 30/40; G16H 50/50

(57) ABSTRACT

The invention relates to a method for visualizing and modeling a dental prosthesis by means of a virtual 3D model of the dental prosthesis, wherein 2D image data, for example x-ray images, are provided. Unavailable information regarding one or more originally present natural teeth is reconstructed by using an algorithm of a deep learning model, by means of which algorithm the virtual 3D model for the dental prosthesis of the patient is generated by derivation from 2D comparison image data, in particular real data sets of comparative persons.

### CROSS REFERENCE TO RELATED APPLICATIONS

This application is a U.S. National Phase application under 35 U.S.C. § 371 of International Application No. PCT/EP2023/061808, filed on May 4, 2023, and claims benefit to German Patent Application No. DE 10 2022 113 599.3 filed on May 30, 2022 and German Patent Application No. DE 10 2023 104 576.8, filed on Feb. 24, 2023. The International Application was published in German on Dec. 7, 2023 as WO 2023/232384 A1 under PCT Article 21(2).

### FIELD

The present disclosure relates to a method for visualizing and modelling dental prostheses using a virtual three dimensional (3D) model of the dental prosthesis, whereby two dimensional (2D) image data, for example X-ray images, are provided. Furthermore, the present disclosure relates to a method for fabricating dental prostheses for a patient based on at least one data set of a virtual 3D model.

### BACKGROUND

In dental practice, impressions are often taken of a patient's upper and lower jaws using plastic hardening impression and/or molding materials in order to obtain an impression. The impression serves as the basis for producing a model. A dental technician molds the dental prosthesis on the model, which is usually configured as a wax model. The wax model usually serves as a positive mold for a negative mold, from which the dental prosthesis is obtained in the well-known artisanal manner and then fitted for the patient and inserted.

Once a patient has lost several teeth, the information about the original anatomy of these teeth and the surrounding bone is lost forever. With the loss of teeth, the information about the original natural occlusion of the teeth and their position and orientation in relation to each other is also lost. This naturally created situation of the two jaws biting together in turn also encodes a precise positional situation of the temporomandibular joint complex and can no longer be determined in this case.

After the loss of several teeth, this anatomical information is lost to varying degrees. The information of an individual tooth in terms of shape and color, the position of the tooth in relation to other teeth, the surrounding jaw shape and the positional relationship of the temporomandibular joint in the intercuspal position can be permanently lost after the loss of the teeth.

The fabrication of dental prostheses by a dentist and/or dental technician aims to reconstruct this lost information and to produce the prosthesis as a replica or as an optimization of the natural teeth and jaw parts as accurate as possible.

Current state-of-the-art procedures are based on the use of reference values of other points of still existing anatomical structures and the use of the experience and theoretical knowledge of the dentist and dental technician in order to come as close as possible to the original shape and position. The final result is therefore largely open in its final appearance. If the quality of a dental prosthesis is measured by the extent to which it corresponds to the original teeth, the current state-of-the-art procedures are not suitable for achieving predictable quality, as the work is largely carried out without knowledge of the original shapes.

In principle, there are many procedures related to the intra-oral cavity in which an accurate three-dimensional virtual representation of the intra-oral cavity can be useful for the practicing dentist.

Such virtual representations (also referred to herein synonymously as “virtual models”, “computer models”, “3D numerical units”, etc.) allow the user to capture the intra-oral cavity and/or inner oral cavity of individual patients via a computer system similar to the examination of the conventional mechanical plaster model.

In dentistry, three-dimensional imaging methods, for example three-dimensional X-rays, magnetic resonance imaging (MRI), three-dimensional computer tomography (CT) or digital volume tomography (DVT), for example as described in DE 10 2008 009 643 A1 or DE 10 2004 035 475 A1, are known for generating medical 3D data sets.

In a method for fabricating dental prostheses according to EP 2 010 090 B1, a measurement data set of a 3D X-ray image is provided in the region of the prosthesis to be used and displayed on a display unit as a 3D X-ray model.

The description of WO 2012/000 511 A1 relates to a method for visualizing and modelling a denture during dental restoration as well as the provision of a virtual 3D model of the denture. For this purpose, firstly 2D images, for example X-ray images, with at least one facial feature are provided, and secondly a virtual 3D model of at least a part of the patient's oral cavity is generated by scanning a physical model or the patient's teeth. Subsequently, the 2D images are aligned relative to the virtual 3D model in a virtual 3D space using detected corresponding anatomical points and visualized in the 3D space. In addition, a dental technician can digitally design or model a restoration on the virtual 3D model using experience and knowledge of dental esthetics and rules.

Furthermore, a method for processing an X-ray image is known from EP 2 509 507 B1, wherein an ultrasonic probe is detected in the 2D X-ray image, and the position and orientation of the ultrasonic probe is estimated in relation to a reference coordinate system.

In practice, it has proven to be disadvantageous that the 3D data required for an exact reconstruction of the lost tooth, unlike X-ray images, is often not available for the fabrication of the denture. In such cases, it is often left to the skill of the treating dentist or dental technician to produce a suitable tooth shape based on experience and, if necessary, to optimize it in the course of repeated adjustments.

This adjustment process is time-consuming and uncomfortable for the patient. However, it has also been shown that, particularly in the case of a large prosthesis replacing several or a large number of teeth, deviations and tolerances that cannot be ruled out in practice can lead to subsequent problems such as pressure pain, impairment of neighboring teeth, pain in the facial and jaw area, clicking or rubbing noises in the jaw joints, above-average wear of the teeth, neck and back problems and even postural problems. Lisping, whistling noises or a change in voice can also occur.

### SUMMARY

In an embodiment, the present disclosure provides a method for visualizing and modelling dental prostheses for a patient based on at least one data set of a virtual three dimensional (3D) model, where two dimensional (2D) image data from 2D X-ray images of originally existing natural teeth of the patient are used, and where an algorithm is used to generate the virtual 3D model for the patient's dental prosthesis based on comparing the 2D image data and 2D comparative image data, where the 2D comparative image data includes real data sets of comparison persons and/or synthetically generated virtual 3D data sets, and 3D comparative data assigned to the 2D comparative image data of at least individual teeth or oral cavity regions.

In an exemplary embodiment, the present disclosure provides a method for fabricating dental prostheses for a patient based on at least one data set of a virtual three dimensional (3D) model, where two dimensional (2D) image data from 2D X-ray images of the patient's originally existing natural teeth are used, where an algorithm is used to generate the virtual 3D model for the patient's dental prosthesis based on comparing the 2D image data and 2D comparative image data of comparison persons, and 3D comparative data assigned to the 2D comparative image data of at least individual teeth or oral cavity regions, where the dental prosthesis and/or a physical model of the dental prosthesis is fabricated using the virtual 3D model.

In some implementations, an optimal match of the 2D image data of the patient with the 2D comparative image data is determined based on a comparative analysis and the 3D comparative data linked to the 2D comparative image data is used for creating the virtual 3D model for the patient's dental prosthesis.

In some implementations, matches and/or deviations of the 2D image data of the patient and the 2D comparative image data are determined in an automated iterative process.

In embodiments, the dental prosthesis and/or a physical functional model is produced based on the virtual 3D model being used in an automated process, where the automated process includes an additive or subtractive fabricating process.

In some implementations, a Computer-Aided Design-Computer-Aided Manufacturing (CAD-CAM) software is used in the fabrication of the dental prosthesis and/or the physical functional model.

In embodiments, the 2D image data obtained from the 2D X-ray images are transferred as an image of teeth to be replaced to a data memory of a cloud or a server.

In some implementations, the algorithm is generated based on a shape analysis and correspondence finding of a training data volume from computer tomography (CT) and digital volume tomography (DVT) data sets.

In embodiments, the virtual 3D model and a 3D model data set of an actual state are spatially referenced to each other using a correspondence finding procedure.

In some implementations, a portion to be replaced as the dental prosthesis is derived based on a differential image analysis of the virtual 3D model and a 3D data set of a current state of the patient.

In another exemplary embodiment, the present disclosure provides a method for generating a virtual three dimensional (3D) model for a patient's dental prosthesis, where the 3D model is generated by an algorithm using two dimensional (2D) comparison image data of comparison persons and linked 3D comparison data.

### BRIEF DESCRIPTION OF THE DRAWING

Subject matter of the present disclosure will be described in even greater detail below based on the exemplary FIGURE. All features described and/or illustrated herein can be used alone or combined in different combinations. The features and advantages of various embodiments will become apparent by reading the following detailed description with reference to the attached drawing, which illustrate the following:

The FIGURE depicts a flowchart for an example of a method for the fabrication of dental prostheses by the dentist or the dental technician in accordance with embodiments of the present disclosure.

### DETAILED DESCRIPTION

The present disclosure describes features for fabricating a dental prosthesis that has no or almost no recognizable deviations from an original natural tooth or only desired optimizations or modifications.

According to the present disclosure, a method is thus provided in which the dental prosthesis for a patient is visualized, modelled and/or fabricated on the basis of at least one data set of a virtual 3D model, where 2D image data, for example from existing 2D X-ray images of the patient's natural teeth originally present to be replaced, are used by comparing the 2D image data with 2D comparison image data of selected or random statistical comparison persons, where the 3D comparison data associated with the 2D comparison image data at least for individual teeth or oral cavity regions are known and/or stored in a database, so that an optimum match of the 2D image data with individual 2D comparison image data is determined on the basis of a comparative observation and the 3D comparison data linked with the 2D comparison image data are used as the basis for creating the 3D model for the patient, where in the case of several matches the selection can be made on the basis of statistical data and additional matching features or an interpolation can be carried out.

According to an aspect of the present disclosure, the dental prosthesis and/or a physical functional model can be fabricated in physical form in this way based on the virtual 3D model created in this way as a data set in an automated additive or subtractive fabricating process.

The present disclosure describes creating a virtual 3D model of the required dental prosthesis solely based on 2D image data, i.e. missing 3D data of the tooth to be replaced, thereby solving problems known from the prior art.

In a high predictable quality, for example, a template for a subsequent dental prosthesis can be calculated by utilizing old panoramic X-ray images of the patient as a 3D model in a fully automated process. This 3D model as template can then be imported into a dental laboratory's Computer-Aided Design-Computer-Aided Manufacturing (CAD-CAM) software using methods known from the prior art and transferred to the patient's natural original situation in the new dental prosthesis.

Contrary to the prevailing prejudice among experts that without the availability of individual 3D data of the tooth structure to be replaced, an identical or almost identical reproduction of prostheses is impossible and therefore also possible unpleasant or even problematic consequences for the patient cannot be reliably avoided, the present disclosure enables generation of a largely identical reproduction of the lost tooth on the basis of the typically available 2D X-ray images, and thus noticeably improves a patient's situation.

According to an exemplary embodiment of the present disclosure, the 2D image data obtained from the X-ray image is uploaded as an image of the teeth to be replaced by the user, particularly the dentist, to a data storage device, for example a cloud or a server. In embodiments, the process thus begins with the import of the so-called panoramic X-ray image of the patient.

An algorithm is used for the reconstruction of the 3D model from the patient's 2D X-ray image. This algorithm uses the correlation of shapes from 2D X-ray images to corresponding 3D shapes. From this shape analysis and correspondence finding of a training data amount from CT and Cone Beam Computed Tomography (CBCT) data sets the algorithm is created and can thus be continuously optimized.

After importation of the panoramic X-ray image, this algorithm is used to generate a corresponding 3D model from the X-ray image as a file, for example in the stereolithography (stl) file format referred to herein as a “.stl file format.” This .stl file of the patient can then be downloaded from the data storage in a dental laboratory by a dental technician and imported into their CAD-CAM software.

According to an exemplary implementation, the dentist's 3D model data set is additionally imported into the dental laboratory's CAD-CAM program after the intra-oral scan of the patient as the patient's individual current state. The virtually created 3D model and the 3D model data set of the current state are then spatially referenced to each other, by means of a correspondence finding procedure, and linked to each other in this way. Thereby, at least three identical points are determined in both model images. These three identical points are used to superimpose the two model data sets.

The dental technician can copy the missing teeth, tooth parts, or jaw parts onto the data set of the intra-oral scan with the missing teeth or jaw parts using the template of the original shape of the teeth and surrounding jaw parts.

Thus, the part to be replaced can be derived as a dental prosthesis based on a differential image analysis by subtracting the current data set (intra-oral scan) from the target data set (template). The dental prosthesis to be produced is therefore not pre-determined before the 3D model was created for the patient but is determined after the model has been created on the basis of the comparative analysis of the corresponding 3D model of the actual state.

This new differential data set then reflects the lost tooth and jaw parts to be restored and corresponds to the dental prosthesis to be fabricated. If necessary, the dental technician can still modify or optimize the original naturally created shape if the patient or dentist wants or needs this change.

The dental prosthesis is then fabricated in the form of the finished data set using state-of-the-art CAM processes from a wide variety of materials.

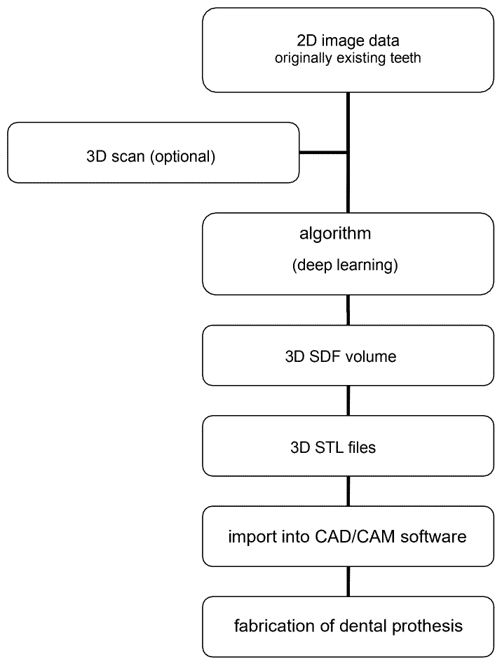

The present disclosure allows for various embodiments. For further clarification, one of these is illustrated in more detail using a flow chart of the method according to the present disclosure and as depicted in FIG. 1.

The method according to the present disclosure is used for the fabrication of dental prostheses by the dentist or the dental technician. For this purpose, non-existent information of one or more originally existing natural teeth is reconstructed so that the dental prosthesis can be fabricated as an exact copy or in the form of an optimization or modification of the natural teeth and jaw parts.

The virtual 3D model for the patient's dental prosthesis is generated in a highly predictable and reproducible quality by utilizing available 2D image data, which may be in the form of previous panoramic X-ray images of the patient's original natural teeth.

Based on the existing and/or available 2D image data of the X-ray image, which contain an image of the teeth to be replaced, whereby a complete set of teeth is not required for the reconstruction, the 2D image data of the patient's X-ray image and, if applicable, a current 3D scan of the patient, created by any intra-oral scanner, as well as, if applicable, other metadata such as gender or age, are initially fed to an algorithm in a first process step. The required inputs for the algorithm may be fed to a scalable web platform.

The algorithm is based on the training of a deep learning model using real and synthetically generated data sets. Thereby, after inputting the 2D X-ray image in the form of a pixel tensor into an auto-encoder-based deep learning architecture, the synthetic data sets are generated based on CT and DVT data sets and 3D models.

If no X-ray information is available in the respective training set, an artificial 2D X-ray image of the respective 3D model is generated via a computer-simulated irradiation and used as an additional training point.

Subsequently, the deep learning model is fine-tuned with real data sets and a 3D SDF volume is created, which is converted into a 3D tensor using a Marching Cubes algorithm, for example, and finally into a 3D triangle mesh, which is exported as an STL file (.stl file).

The dental technician or the dentist can then download this final, reconstructed STL file from the web platform, for example, and import it into CAD-CAM software. The current patient situation can also be imported into the CAD-CAM program as a 3D model data set after the intra-oral scan of the patient.

Both the reconstructed 3D data set and the patient's current situation are linked with each other using a Many-to-Many point matching process. Thereby, at least three identical points are determined in both model images. These at least three identical points are used to superimpose, scale, and transform both model data sets.

Using the template of the original shape of the teeth and surrounding jaw parts, the dental technician or the dentist can copy the missing teeth, tooth portions, or jaw portions onto the data set of the intra-oral scan with the missing teeth or jaw parts. This new data set then reflects the lost tooth parts and jaw parts to be restored. This file corresponds to the dental prosthesis to be fabricated.

If necessary, the original, naturally created shape can be modified or optimized based on the patient or the dentist wishes, or if it is required.

The dental prosthesis is then fabricated from the desired material based on this final data set using CAM procedures.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for visualizing and modelling dental prostheses for a patient based on at least one data set of a virtual three dimensional (3D) model,
wherein two dimensional (2D) image data from 2D X-ray images of originally existing natural teeth of the patient are used, and
wherein an algorithm is used to generate the virtual 3D model for the patient's dental prosthesis based on comparing the 2D image data and 2D comparative image data, in particular wherein the 2D comparative image data includes real data sets of comparison persons and/or synthetically generated virtual 3D data sets, and 3D comparative data assigned to the 2D comparative image data of at least individual teeth or oral cavity regions.

2. A method for fabricating dental prostheses for a patient based on at least one data set of a virtual three dimensional (3D) model,
wherein two dimensional (2D) image data from 2D X-ray images of the patient's originally existing natural teeth; are used,
wherein an algorithm is used to generate the virtual 3D model for the patient's dental prosthesis based on comparing the 2D image data and 2D comparative image data of comparison persons, and 3D comparative data assigned to the 2D comparative image data of at least individual teeth or oral cavity regions, and
wherein the dental prosthesis and/or a physical model of the dental prosthesis is fabricated using the virtual 3D model.

3. The method according to claim 1, wherein an optimal match of the 2D image data of the patient with the 2D comparative image data is determined based on a comparative analysis and the 3D comparative data linked to the 2D comparative image data is used for creating the virtual 3D model for the patient's dental prosthesis.

4. The method according to claim 1, wherein matches and/or deviations of the 2D image data of the patient and the 2D comparative image data are determined in an automated iterative process.

5. The method according to claim 1, wherein the dental prosthesis and/or a physical functional model is produced based on the virtual 3D model being used in an automated process, wherein the automated process includes an additive or subtractive fabricating process.

6. The method according to claim 5, wherein a Computer-Aided Design-Computer-Aided Manufacturing (CAD-CAM) software is used in the fabrication of the dental prosthesis and/or the physical functional model.

7. The method according to claim 1 wherein the 2D image data obtained from the 2D X-ray images are transferred as an image of teeth to be replaced to a data memory of a cloud or a server.

8. The method according to claim 1, wherein the algorithm is generated based on a shape analysis and correspondence finding of a training data volume from computer tomography (CT) and digital volume tomography (DVT) data sets.

9. The method according to claim 1, wherein the virtual 3D model and a 3D model data set of an actual state are spatially referenced to each other using a correspondence finding procedure.

10. The method according to claim 1, wherein a portion to be replaced as the dental prosthesis is derived based on a differential image analysis of the virtual 3D model and a 3D data set of a current state of the patient.

11. A method for generating a virtual three dimensional (3D) model for a patient's dental prosthesis, wherein the 3D model is generated by an algorithm using two dimensional (2D) comparison image data of comparison persons and linked 3D comparison data.
